(19) United States
(12) Patent Application Publication
(10) Pub. No.: US 2021/0093883 A1
(43) Pub. Date: Apr. 1, 2021

(54) LIGHT IRRADIATION DEVICE AND LIGHT IRRADIATION METHOD

(71) Applicants: Public University Corporation Nagoya City University, Nagoya-city, Aichi (JP); Ushio Denki Kabushiki Kaisha, Chiyoda-ku, Tokyo (JP)

(72) Inventors: Jun MORI, Osaka (JP); Katsuji IGUCHI, Osaka (JP); Hitoshi AOKI, Osaka (JP); Akimichi MORITA, Aichi (JP); Hideyuki MASUDA, Tokyo (JP); Makoto KIMURA, Tokyo (JP)

(21) Appl. No.: 17/050,766

(22) PCT Filed: Apr. 5, 2019
(86) PCT No.: PCT/JP2019/015222

§ 371 Date: Oct. 26, 2020

(30) Foreign Application Priority Data
Apr. 27, 2018 (JP) 2018-087643

(51) Int. Cl. A61N 5/06

(52) U.S. Cl. CPC A61N 5/062; A61N 2005/0647; A61N 5/0616

(57) ABSTRACT

An object is to uniformly irradiate a non-flat affected area with light. Provided is an irradiation apparatus (1) configured to irradiate a projecting part of a body of an irradiation target organism with light, the irradiation apparatus including: a cover (2); a light source unit (3); and a sheet (4) that is provided so that the light source unit (3) does not make contact with an affected area of the projecting part or an area surrounding the affected area, the sheet (4) and/or the cover (2) being configured to make contact with the area surrounding the affected area.

### TECHNICAL FIELD

The present invention relates to an irradiation apparatus and a method of irradiation.

### BACKGROUND ART

One of the conventionally known therapies using light is a photodynamic therapy (hereinafter referred to as “PDT”). PDT is a therapy to selectively destroy only a lesion (lesion/abnormal tissue) in the following manner, making use of the properties of a photosensitizing substance having an affinity for a lesion of a living body (specifically, making use of the property of accumulating specifically in the lesion): the photosensitizing substance or a photosensitizing substance precursor is administered to the living body; thereafter the photosensitizing substance (including a photosensitizing substance synthesized form the photosensitizing substance precursor in the living body) is irradiated with light; and reactive oxygen species generated in the tissue is used to selectively destroy only the lesion/abnormal tissue. Such PDT is a minimally invasive therapy which does not damage normal cells, and is therefore attracting great attention these days in terms of quality of life (QOL). In recent years, in the field of dermatology, PDT has been widely used to treat, for example: tumorous lesions such as actinic keratosis, Bowen's disease, Paget's disease, and basal cell cancer; severe acne vulgaris; sebaceous hyperplasia; and intractable warts.

Actinic keratosis is characterized in that it develops in sun-exposed areas and therefore frequently develops on the face, fingers, and the like. With regard to the face, especially the nose and ears are parts protruding to a relatively great extent. Other than the face, fingers are parts protruding to a relatively great extent. (Such parts hereinafter may be referred to as “projecting parts”.) Each projecting part is also characterized in that it differs greatly among individuals. Other dermatological diseases are also characterized in that they develop on projecting parts.

Under such circumstances, there is a demand for an irradiation apparatus that is smaller in size and that is capable of easily carrying out PDT on projecting parts (an irradiation apparatus for use in PDT hereinafter may be referred to as PDT irradiation apparatus).

Known PDT irradiation apparatuses are, for example, apparatuses including a lamp-type light source such as LED, halogen lamp, xenon lamp, or metal halide lamp.

### CITATION LIST

### Patent Literature

[Patent Literature 1]

Japanese Patent Application Publication, Tokukai, No. 2017-6454 (Publication date: Jan. 12, 2017)

[Patent Literature 2]

Published Japanese Translation of PCT International Application, Tokuhyo, No. 2002-511323 (Publication Date: Apr. 16, 2002)

[Patent Literature 3]

Japanese Patent No. 5763627 (Registration Date: Jun. 19, 2015)

### SUMMARY OF INVENTION

### Technical Problem

However, the foregoing conventional irradiation apparatuses have the following issues.

A lesion is placed at a certain distance from or at a certain angle to a light source which is in a fixed position, and irradiated with intense light (for example, 50 J/cm2). Therefore, in a case where an apparatus including a lamp-type light source is used with respect to a projecting part, the patient may need to take an uncomfortable posture depending on which portion of the projecting part, i.e., front side, back side, or lateral side of the projecting part, is to be irradiated.

Furthermore, in a case where a conventional PDT irradiation apparatus is used to irradiate, with light, a lesion on the nose or an ear each of which is one of the projecting parts, medical staff and the patient need to wear sunglasses or the like in order to prevent the intense light emitted by the apparatus from entering the eyes. This is troublesome.

In addition, an ear, which is one of the projecting parts, has front and back sides. In a case where a conventional PDT irradiation apparatus is used to irradiate a lesion on the ear with light, it is necessary to, for example, bend the ear of the patient. This takes time.

Note that several techniques have been proposed to respond to the above-stated demands.

Patent Literature 1 discloses an irradiation apparatus for use in photodynamic therapy, which includes a light source unit made up of first LED elements (having a peak wavelength at 400 nm to 420 nm) and second LED elements (having a peak wavelength at 500 nm to 520 nm) arranged alternately in a grid pattern. Both the first LED elements and the second LED elements are turned ON with respect to a single part to be irradiated, and thereby the part is irradiated with light from the first LED elements and light from the second LED elements. However, Patent Literature 1 does not disclose how the apparatus is fitted to a projecting part which greatly differs among individuals.

Patent Literature 2 discloses a technique of placing light sources in an interspersed manner on a substrate that corresponds in shape to the body, and discloses that uniform irradiation is achieved. However, Patent Literature 2 does not disclose how the light sources are fixed to the body. Furthermore, the technique disclosed in Patent Literature 2 necessitates preparation of a substrate and light sources that correspond in size and shape to the body, which costs a lot.

Patent Literature 3 discloses a technique in which light sources are contained in a cover, and discloses that a phototherapy apparatus can be attached to the body and a lesion can be irradiated with light. Patent Literature 3 does not disclose how the phototherapy apparatus is fitted to a projecting part that greatly differs among individuals. Patent Literature 3 does not disclose any technique to achieve uniform irradiation, either.

Furthermore, when an individual wearing the PDT irradiation apparatus moves, the lesion or the projecting part may touch the light source in the apparatus, and an undesirable event such as a burn may result. None of the foregoing Patent Literatures 1, 2 and 3 discloses any means to solve such issues.

An aspect of the present invention was made in view of the foregoing issues, and an object thereof is to uniformly irradiate a projecting part with light. Another object is to efficiently and safely irradiate, with light, a projecting part which differs greatly among individuals.

### Solution to Problem

In order to attain the above object, an irradiation apparatus in accordance with an aspect of the present invention is an irradiation apparatus configured to irradiate a projecting part of a body of an irradiation target organism with light, the irradiation apparatus including: a cover that corresponds in shape to the projecting part; a light source unit that is superposed on the cover; and a sheet that is provided so that the light source unit does not make contact with an affected area of the projecting part or an area surrounding the affected area and that is superposed on the cover with the light source unit interposed between the sheet and the cover, the sheet and/or the cover being configured to make contact with the area surrounding the affected area.

### Advantageous Effects of Invention

An aspect of the present invention makes it possible to uniformly irradiate a projecting part with light, and possible to efficiently and safely irradiate a projecting part with light.

### BRIEF DESCRIPTION OF DRAWINGS

FIG. 1 is a front view schematically illustrating a configuration of an irradiation apparatus in accordance with Embodiment 1 of the present invention.

FIG. 2 is a first cross-sectional view schematically illustrating the configuration of the irradiation apparatus in accordance with Embodiment 1 of the present invention.

FIG. 3 is a second cross-sectional view schematically illustrating the configuration of the irradiation apparatus in accordance with Embodiment 1 of the present invention.

FIG. 4 is a cross-sectional view schematically illustrating a configuration of an example of a light source unit in accordance with the present invention.

FIG. 5 is a cross-sectional view schematically illustrating a configuration of an irradiation apparatus in accordance with Embodiment 2 of the present invention.

FIG. 6 is a cross-sectional view schematically illustrating a configuration of an irradiation apparatus in accordance with Embodiment 3 of the present invention.

FIG. 7 is a cross-sectional view schematically illustrating a configuration of an irradiation apparatus in accordance with Embodiment 4 of the present invention.

FIG. 8 is a cross-sectional view schematically illustrating a configuration of an irradiation apparatus in accordance with Embodiment 5 of the present invention.

FIG. 9 is a cross-sectional view schematically illustrating a configuration of an irradiation apparatus in accordance with Embodiment 6 of the present invention.

FIG. 10 is a cross-sectional view schematically illustrating a configuration of an irradiation apparatus in accordance with Embodiment 7 of the present invention.

FIG. 11 is a cross-sectional view schematically illustrating a configuration of an irradiation apparatus in accordance with Embodiment 8 of the present invention.

FIG. 12 is a cross-sectional view schematically illustrating a configuration of an irradiation apparatus in accordance with Embodiment 9 of the present invention.

FIG. 13 is a front view schematically illustrating a configuration of an irradiation apparatus in accordance with Embodiment 10 of the present invention.

FIG. 14 is a front view schematically illustrating a configuration of an irradiation apparatus in accordance with Embodiment 11 of the present invention.

FIG. 15 is a cross-sectional view schematically illustrating a configuration of an irradiation apparatus in accordance with Embodiment 12 of the present invention in the vicinity of the eyes.

FIG. 16 is a cross-sectional view schematically illustrating a configuration of an irradiation apparatus in accordance with Embodiment 13 of the present invention in the vicinity of the eyes.

FIG. 17 is a top view schematically illustrating a configuration of an irradiation apparatus in accordance with Embodiment 14 of the present invention.

FIG. 18 is a first cross-sectional view schematically illustrating the configuration of the irradiation apparatus in accordance with Embodiment 14 of the present invention.

FIG. 19 is a second cross-sectional view schematically illustrating the configuration of the irradiation apparatus in accordance with Embodiment 14 of the present invention.

FIG. 20 is a perspective view schematically illustrating the configuration of the irradiation apparatus in accordance with Embodiment 14 of the present invention.

FIG. 21 is a perspective view schematically illustrating a configuration of an irradiation apparatus in accordance with Embodiment 15 of the present invention.

FIG. 22 is a cross-sectional view schematically illustrating the configuration of the irradiation apparatus in accordance with Embodiment 15 of the present invention.

FIG. 23 is a top view schematically illustrating a configuration of an irradiation apparatus in accordance with Embodiment 16 of the present invention.

FIG. 24 is a first cross-sectional view schematically illustrating the configuration of the irradiation apparatus in accordance with Embodiment 16 of the present invention.

FIG. 25 is a second cross-sectional view schematically illustrating the configuration of the irradiation apparatus in accordance with Embodiment 16 of the present invention.

FIG. 26 is a cross-sectional view schematically illustrating a configuration of an irradiation apparatus in accordance with Embodiment 17 of the present invention.

### DESCRIPTION OF EMBODIMENTS

The following description will discuss embodiments of the present invention in detail. Note, however, that the sizes, materials, shapes, relative positions, method of processing, and the like of constituents described in the following embodiments are merely examples, and should not be interpreted as limiting the scope of the present invention. Also note that the drawings are schematic drawings, and relative sizes and shapes are different from actual ones.

Note that, in the present specification, an “irradiation target organism” is not limited to a human. For example, animals are also included in the definition of the “irradiation target organism”.

In the present specification, a “projecting part” means a part that protrudes to a relatively great extent, such as nose, ear, finger, and male genital organ. Explanations made using one of such parts as an example are applicable to other parts. Furthermore, the projecting part is characterized in that it differs greatly among individuals.

Also note that specific details of an agent for use in PDT, wavelengths of light, and the like have no effect on the configuration of an irradiation apparatus in accordance with an aspect of the present invention, and therefore are not discussed in the following descriptions.

### Embodiment 1

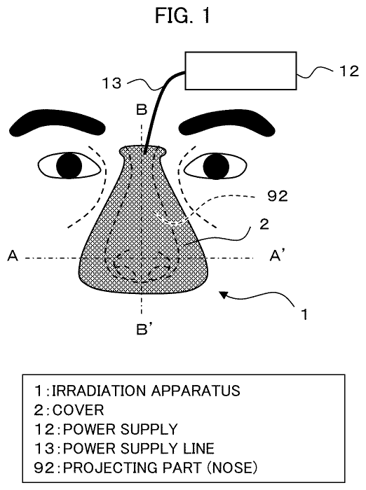

The following description will discuss Embodiment 1 of the present invention with reference to FIGS. 1 to 3.

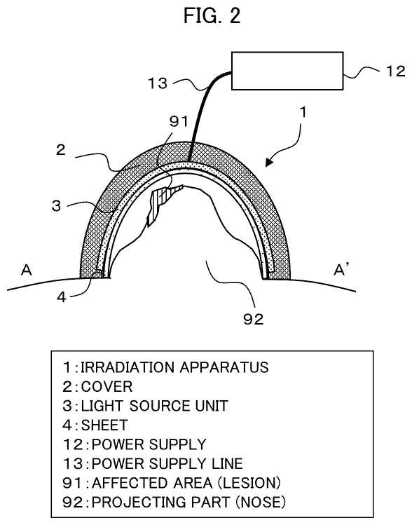

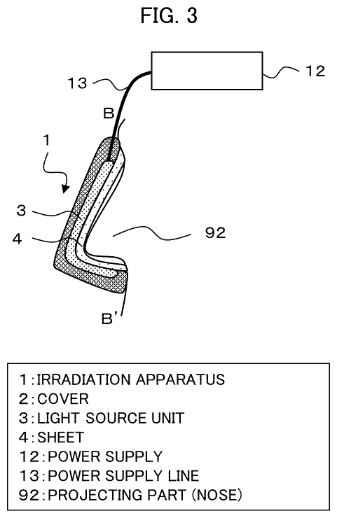

FIG. 1 is a front view schematically illustrating a configuration of an irradiation apparatus 1. FIG. 2 is a first cross-sectional view schematically illustrating the configuration of the irradiation apparatus 1. FIG. 3 is a second cross-sectional view schematically illustrating the configuration of the irradiation apparatus 1.

FIG. 2 is a cross-sectional view of the irradiation apparatus 1 taken along line A-A′ in FIG. 1. Similarly, FIG. 3 is a cross-sectional view of the irradiation apparatus 1 taken along line B-B′ in FIG. 1.

### Overview of Configuration of Irradiation Apparatus 1

The following description will discuss an overview of the configuration of the irradiation apparatus 1 in accordance with Embodiment 1 of the present invention with reference to FIGS. 1 to 3.

This PDT irradiation apparatus is for use in carrying out photodynamic therapy in the following manner: a substance for administration to a living body, composed of a photosensitizing substance or a photosensitizing substance precursor, is administered to the living body; and thereafter the photosensitizing substance (including a photosensitizing substance synthesized form the photosensitizing substance precursor in the living body) accumulated in a lesion (lesion/abnormal tissue) is irradiated with light.

The substance for administration to a living body is, for example, a compound that reacts in the living body as necessary and accumulates as a porphyrin compound in the lesion.

Specific examples of the substance for administration to a living body include δ-aminolevulinic acid (5-ALA). The δ-aminolevulinic acid is a photosensitizing substance precursor, from which protoporphyrin IX (PpIX) is synthesized via enzyme reaction and functions as a photosensitizing substance.

When the substance for administration to a living body is topically administered (applied), the substance is applied over an affected area and its surrounding area of about several millimeters, in many cases.

As illustrated in FIGS. 1 to 3, the irradiation apparatus 1 includes a cover 2, a light source unit 3, a sheet 4, a power supply 12, and a power supply line 13. As illustrated in FIGS. 2 and 3, the cover 2 has, in its surface facing an affected area (lesion) 91 and a projecting part (nose) 92, a place for disposition of the light source unit 3. The light source unit 3 is disposed in this place, and the sheet 4 covers the light source unit 3. Note that the cover 2 does not need to have the place for disposition of the light source unit 3; however, the presence of this place makes it possible to more easily equip the cover 2 with the light source unit 3. An adhesive can be applied to the place for disposition of the light source unit 3 in advance, an adhesive can be applied between the light source unit 3 and the cover 2, and/or adhesive tape can be placed between the light source unit 3 and the cover 2.

The cover 2 and the sheet 4 are each in partial contact with the projecting part (nose) 92. Therefore, the cover 2 and the sheet 4 are each preferably composed of a material that is safe for the body of an irradiation target organism (such as the projecting part (nose) 92) to touch.

The light source unit 3 is connected to the power supply 12 through the power supply line 13. The power supply 12 may be connected to, for example, an electrical outlet for medical use. The power supply line 13 is connected to a part of the light source unit 3, and passes through the cover 2 via a through hole. Note that, in order to prevent intense light emitted by the light source unit 3 from passing through the through hole and entering the eyes of the irradiation target organism and medical staff, it is preferable that the through hole be covered with or filled with a material that is non-transparent to the light emitted by the light source unit 3. Note that such a manner of connection is preferred in a case where, for example, the power supply 12 is worn on the body.

The power supply line 13 may be passed between the cover 2 and the projecting part (nose) 92. In this case, it is possible to most easily produce the apparatus. Note that the power supply line 13 is preferably made from a material that is safe for skin contact.

In FIG. 1, the power supply line 13 is run outward in a direction toward the forehead; however, the power supply line 13 may be run along a cheek or may be run downward from the nose. In a case where the power supply line 13 is run along a cheek or is run downward from the nose, light leakage which would result from the power supply line 13 run outward is ignorable to the irradiation target organism. This is because most of such leaked light does not enter the eyes of the patient. Therefore, in such a case, it is possible to relatively easily produce the apparatus.

Note that the power supply line 13 may have a connector connected thereto. The connector may be configured to allow the light source unit 3 and the power supply 12 to be easily disconnected by a certain amount of force. This makes it possible, in a case where the irradiation target organism moved violently in response to, for example, a sudden pain, to easily stop the emission of light from the light source unit 3 and discontinue the therapy.

Note that the power supply 12 and the power supply line 13 are not illustrated in in the subsequent drawings. In the descriptions with reference to the subsequent drawings, the foregoing descriptions for the power supply 12 and the power supply line 13 will not be repeated.

The following description will discuss each constituent of the irradiation apparatus 1 in more detail.

### Cover 2

The cover 2 covers at least the affected area (lesion) 91, as illustrated in FIGS. 1 to 3. The cover 2 further entirely covers the projecting part (nose) 92. This is because the affected area (lesion) 91 can develop in any area of the projecting part (nose) 92. The cover 2 is preferably larger in size than the maximum size of the projecting part (nose) 92. If the cover 2 is smaller in size than the maximum size of the projecting part (nose) 92, there may be cases in which the projecting part (nose) 92 cannot be covered by the cover 2 and PDT cannot be carried out appropriately. A plurality of types of covers 2 may be prepared in advance.

The cover 2 can make contact with the affected area (lesion) 91 or the projecting part (nose) 92. Therefore, the cover 2 is preferably composed of a material that is safe for a living body to touch. In some cases, the cover 2 may have a multilayer structure whose surface only is composed of a material that is safe for a living body to touch.

The cover 2 may be produced with use of a 3D printer or the like using 3D data acquired from the affected area (lesion) 91 and the projecting part (nose) 92 with a 3D scanner or the like. This makes it possible to more appropriately produce the cover 2 for the affected area (lesion) 91 even if the affected area (lesion) 91 is unsafe to touch.

The cover 2 is preferably flexible to some extent. This is because such a cover 2 can be changed in shape to some extent to conform to the shape of the projecting part (nose) 92.

The cover 2 may be partially or entirely composed of an elastic material. With this, the irradiation apparatus 1 can be appropriately worn on the projecting part (nose) 92 even if the projecting part (nose) 92 is larger than expected.

Examples of a material that can be used to make the cover 2 include cloth, rubber, plastic, ABS resin, gypsum, nylon resin, acrylic resin, polycarbonate resin, polypropylene resin, silicone resin, epoxy resin, styrene-based elastomer, water-containing polymer gel, and the like. The cover 2 is preferably heat resistant. This is to prevent the cover 2 from deforming due to heat from the light source unit 3.

The cover 2 preferably has insulating property. This is to protect the irradiation target organism and the medical staff against electric current flowing through the light source unit 3. In particular, with use of a water-containing polymer gel, it is possible to cool the heat from the light source unit 3.

The cover 2 may be cooled in a refrigerator or the like in advance. In so doing, the temperature of the surface facing away from the affected area (lesion) 91 is preferably equal to or higher than a temperature that is safe to touch, because this surface may be touched by the irradiation target organism and the medical staff. On the contrary, if the cover is overcooled, the cover 2 will lose its flexibility. The temperature of the cover 2 is therefore preferably not lower than 0° C., more preferably not lower than 10° C. The temperature to which the cover 2 is cooled can be selected appropriately in consideration of the amount of heat emitted by the light source unit 3. The cover 2 may be a plastic bag having therein a cold reserving material, coolant, water, and/or the like.

The cover 2 may have an adhesive portion. This makes it possible to fix the cover 2 to the projecting part (nose) 92, and allows the irradiation target organism to move while PDT is carried out. This also makes it possible to carry out PDT at home.

The cover 2 may be at least partially non-transparent to light. This makes it possible to prevent intense light emitted by the light source unit 3 from directly entering the eyes of the irradiation target organism and the medical staff. However, if the light is completely blocked, there may be cases in which the intense light emitted by the light source unit 3 is completely hidden from view of the irradiation target organism and the medical staff, and whether the light irradiation is appropriately carried out cannot be checked by the irradiation target organism and the medical staff. To address such inconvenience, the cover 2 may have a completely light-blocking portion and a completely light-transparent portion.

### Sheet 4

The sheet 4 serves to prevent the light source unit 3 from making direct contact with the affected area (lesion) 91 and the projecting part (nose) 92. The cover 2 can make contact with the affected area (lesion) 91 or the projecting part (nose) 92.

The sheet 4 is therefore preferably composed of a material that is safe for a living body to touch. Examples of such a material include silicone resin for medical use, epoxy resin, styrene-based elastomer resin, and the like. Note, however, that the material is not limited to the resins listed above, provided that the material has similar characteristics to those of the resins listed above. In some cases, the sheet 4 may have a multilayer structure whose surface only is composed of a material that is safe for a living body to touch.

The sheet 4 should be transparent to light emitted by the light source unit 3. The sheet 4 may be formed by including, in a sheet base material, a diffusing agent which scatters light emitted by the light source unit 3, such as polymethyl methacrylate (PMMA). Alternatively, a light diffusing sheet may be used as the sheet 4. This makes it possible to more uniformly irradiate the affected area (lesion) 91 and the projecting part (nose) 92 with the light emitted by the light source unit 3.

The sheet 4 preferably has heat insulating property. This makes it possible to provide a shield against heat emitted by the light source unit 3, and thus possible to prevent undesirable events, such as a burn, from occurring if the affected area (lesion) 91 and/or the projecting part (nose) 92 make contact with the sheet 4.

The sheet 4 is not limited to those in the form of a molded sheet. The sheet 4 may be formed by, for example, applying a material that is transparent to the light emitted by the light source unit 3.

The sheet 4 is preferably waterproof. This is to prevent an agent used in PDT from entering the light source and causing an electrical failure.

### Light Source Unit 3

The light source unit 3 irradiates the affected area (lesion) 91 with light that meets minimum conditions required for PDT, e.g., light having a light intensity, wavelength, and/or the like required for PDT. The light source unit 3 is preferably a so-called surface-emitting light source. This makes it possible to effectively irradiate the entire projecting part (nose) 92 with light. The surface-emitting light source can be, for example, an organic EL device, two-dimensionally arranged LEDs, or the like.

The light source unit 3 is preferably capable of being bent along the cover 2. The light source unit 3 preferably has a shape that fits the cover 2, and can be bonded to the cover 2 with an adhesive or the like.

Examples of the light source unit 3 that can be employed include light sources using a laser, an LED, an organic EL device, or the like. Note, however, that the light source unit 3 is not limited to those listed above, provided that the light source unit 3 has the above-stated characteristics.

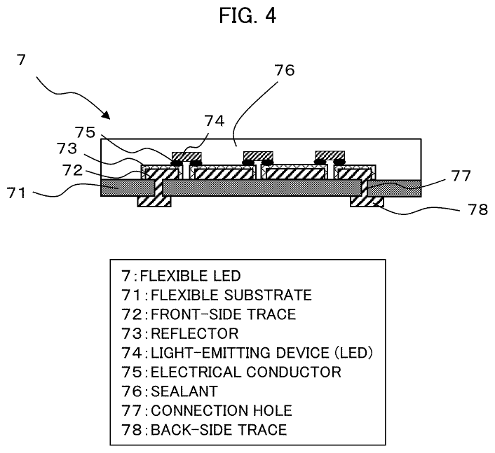

FIG. 4 illustrates a flexible LED 7, which is an example of the light source unit 3. The flexible LED 7 includes a flexible substrate 71, front-side traces 72, reflectors 73, light-emitting devices (LEDs) 74, electrical conductors 75, a sealant 76, connection holes 77, and back-side traces 78.

The flexible substrate 71 has, provided on its first face, the front-side traces 72 made of, for example, copper, and the light-emitting devices (LEDs) 74 are mounted on the front-side traces 72. The front-side traces 72 are covered with the reflectors 73, which are made of a material having high reflectance, for example, a total reflectance of not less than 80%, such as silver. The light-emitting devices (LEDs) 74 are electrically connected to the front-side traces 72 through the electrical conductors 75. The flexible substrate 71, the front-side traces 72, and the light-emitting devices (LEDs) 74 are sealed with the sealant 76. The front-side traces 72 are electrically connected to the back-side traces 78 made of, for example, copper, through the connection holes 77.

For example, the back-side traces 78 are electrically connected to electrodes on the surface of the cover 2 facing toward the affected area (lesion) 91 and the projecting part (nose) 92. With this, when electric current is applied to the electrodes, the light-emitting devices (LEDs) 74 emit light.

The sealant 76 can be a sealing resin such as silicone resin. The sealing resin can have a wavelength conversion material, such as a phosphor, mixed therein.

The following description will discuss each constituent of the flexible LED 7 of FIG. 4 in more detail.

### Flexible Substrate 71

The flexible substrate 71 is a flexible insulating substrate made of a resin material, and is constituted by, for example, a polyimide insulating film.

Note, however, that the material for the flexible substrate 71 is not limited to polyimide. The material for the flexible substrate 71 can be any material, provided that the material is an insulating material that has a necessary mechanical strength and flexibility. Examples of the flexible substrate 71 include not only polyimide resin films but also films made of fluorocarbon resin, silicone resin, polyethylene terephthalate resin, or the like. The flexible substrate 71 can be a highly reflective resin film obtained by applying a white pigment-containing resin (such as white resin or white resist) on the surface of any of the above-listed films, a highly reflective film having a white pigment mixed therein, a liquid crystal polymer film, or the like.

The flexible substrate 71 has a thickness of, for example, 25 μm to 200 μm. In a case where the flexible substrate 71 is too thin, it may be difficult to obtain a desired mechanical strength. On the contrary, in a case where the flexible substrate 71 is too thick, it may be difficult to obtain a required flexibility. That is, with regard to the thickness of the flexible substrate 71, mechanical strength and flexibility are in a trade-off relationship, and there are appropriate values. The thickness of the flexible substrate 71 is more preferably 40 μm to 100 μm.

As has been described, the irradiation apparatus 1 in accordance with Embodiment 1 is an irradiation apparatus 1 configured to irradiate a projecting part (nose) 92 of the body of an irradiation target organism with light, the irradiation apparatus 1 including: a cover 2 that corresponds in shape to the projecting part (nose) 92; a light source unit 3 that is superposed on the cover 2; and a sheet 4 that is provided so that the light source unit 3 does not make contact with an affected area (lesion) 91 of the projecting part (nose) 92 or an area surrounding the affected area (lesion) 91 and that is superposed on the cover 2 with the light source unit 3 interposed between the sheet 4 and the cover 2. The sheet 4 and/or the cover 2 make(s) contact with the area surrounding the affected area (lesion) 91.

### Embodiment 2

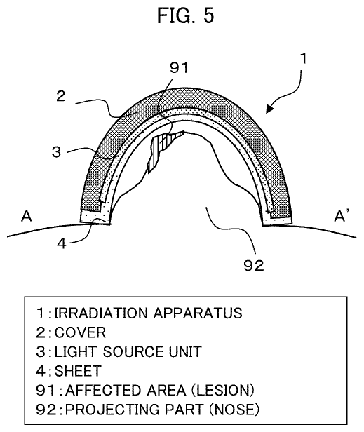

The following description will discuss Embodiment 2 of the present invention with reference to FIG. 5. For convenience of description, members having functions identical to those described in Embodiment 1 are assigned identical referential numerals, and their descriptions are omitted here. In Embodiment 2, differences from Embodiment 1 are discussed.

FIG. 5 is a cross-sectional view illustrating a configuration of an irradiation apparatus 1 in accordance with Embodiment 2.

As illustrated in FIG. 5, the irradiation apparatus 1 includes a cover 2, a light source unit 3, and a sheet 4, similarly to Embodiment 1. Embodiment 2 is different from Embodiment 1 in that only the sheet 4 is in contact with a projecting part (nose) 92. In other words, the sheet 4 partially covers the cover 2 so that the cover 2 does not make contact with the projecting part (nose) 92. Unlike Embodiment 1, the cover 2 does not need to be composed of a material that is safe for the projecting part (nose) 92, i.e., a part of the body of an irradiation target organism, to touch. This can reduce cost.

### Embodiment 3

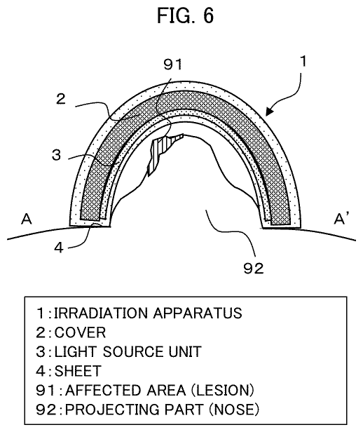

The following description will discuss Embodiment 3 of the present invention with reference to FIG. 6. For convenience of description, members having functions identical to those described in Embodiment 2 are assigned identical referential numerals, and their descriptions are omitted here. In Embodiment 3, differences from Embodiment 2 are discussed.

FIG. 6 is a cross-sectional view illustrating a configuration of an irradiation apparatus 1 in accordance with Embodiment 3.

As illustrated in FIG. 6, the irradiation apparatus 1 includes a cover 2, a light source unit 3, and a sheet 4, and only the sheet 4 is in contact with a projecting part (nose) 92, similarly to Embodiment 2. Embodiment 3 is different from Embodiment 2 in that the entire sheet 4 covers both faces of the cover 2. In Embodiment 3, the sheet 4 can be formed by, for example attaching the light source unit 3 to the cover 2 to obtain an assembly and then forming a coating on the entire surface of the assembly or laminating the assembly. This can reduce cost compared to the foregoing embodiments.

### Embodiment 4

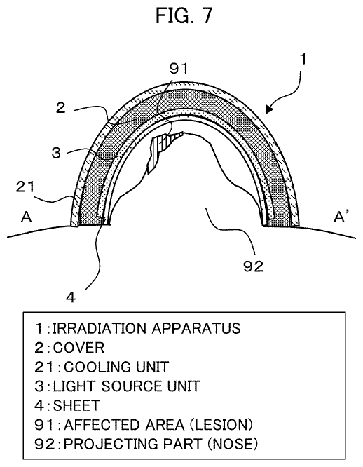

The following description will discuss Embodiment 4 of the present invention with reference to FIG. 7. For convenience of description, members having functions identical to those described in Embodiment 1 are assigned identical referential numerals, and their descriptions are omitted here. In Embodiment 4, differences from Embodiment 1 are discussed.

FIG. 7 is a cross-sectional view illustrating a configuration of an irradiation apparatus 1 in accordance with Embodiment 4.

As illustrated in FIG. 7, the irradiation apparatus 1 includes a cover 2, a light source unit 3, and a sheet 4, similarly to Embodiment 1. The irradiation apparatus 1 further includes a cooling unit 21. The cooling unit 21 can be, for example, a cold reserving material, a fan, a Peltier element, or the like.

The cooling unit 21 is in contact with the surface of the cover 2 which surface faces away from an affected area (lesion) 91. The cover 2 is cooled by the cooling unit 21, and thereby the light source unit 3 is cooled indirectly. The irradiation apparatus 1 in accordance with Embodiment 4 is not arranged such that the cooling unit 21 directly cools the light source unit 3; however, the cooling means is provided on the outer surface of the cover 2 and therefore the arrangement is simple. This makes it possible to attach the cooling unit 21 on the outer surface of the cover 2 at the site where PDT is carried out.

As such, the irradiation apparatus 1 in accordance with Embodiment 4 includes a cooling unit 21 that is configured to cool the light source unit 3 and that is superposed on at least one face of the cover 2.

### Embodiment 5

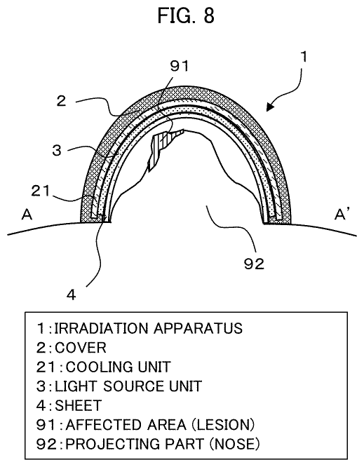

The following description will discuss Embodiment 5 of the present invention with reference to FIG. 8. For convenience of description, members having functions identical to those described in Embodiment 4 are assigned identical referential numerals, and their descriptions are omitted here. In Embodiment 5, differences from Embodiment 4 are discussed.

FIG. 8 is a cross-sectional view illustrating a configuration of an irradiation apparatus 1 in accordance with Embodiment 5. As illustrated in FIG. 8, the irradiation apparatus 1 includes a cover 2, a light source unit 3, and a sheet 4, and further includes a cooling unit 21, similarly to Embodiment 4. Embodiment 5 is different from Embodiment 4 in that the cooling unit 21 is provided on the surface of the cover 2 which surface faces toward an affected area (lesion) 91 or is provided inside the cover 2. Therefore, at least one face of the light source unit 3 and the cooling unit 21 are in direct with each other and efficient heat exchange is achieved, making it possible to reduce the time required for cooling. This makes it possible to carry out PDT efficiently.

Note that, by arranging the cooling unit 21 such that the cooling unit 21 is flexible and such that at least one face of the cooling unit 21 has adhesiveness to allow an object contacting thereto to adhere thereto, it is possible to not only cool the light source unit 3 as illustrated in FIG. 4 but also fix the light source unit 3.

The cooling unit 21 can be arranged to make direct contact with the affected area (lesion) 91 or a projecting part (nose) 92. During PDT, an irradiation target organism may feel hot to some degree. In such a case, the cooling unit 21 contacting the organism is preferred because the organism feels cool and the feeling of heat can be reduced.

On the contrary, for preventing the cooling unit 21 from making direct contact with the affected area (lesion) 91 or the projecting part (nose) 92 of the irradiation target organism, a part of the cooling unit 21 (especially a part not having the light source unit 3 bonded thereto) can be covered by the sheet 4. Such an arrangement is preferred because it is possible to prevent the organism from suffering from accidental frostbite.

### Embodiment 6

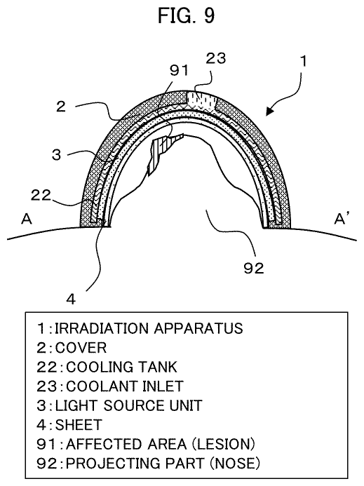

The following description will discuss Embodiment 6 of the present invention with reference to FIG. 9. For convenience of description, members having functions identical to those described in Embodiment 5 are assigned identical referential numerals, and their descriptions are omitted here. In Embodiment 6, differences from Embodiment 5 are discussed.

FIG. 9 is a cross-sectional view illustrating a configuration of an irradiation apparatus 1 in accordance with Embodiment 6.

As illustrated in FIG. 9, the irradiation apparatus 1 includes a cover 2, a light source unit 3, and a sheet 4, similarly to Embodiment 5. A cooling unit 21 further has a coolant inlet 23 and functions as a cooling tank 22.

The cooling tank 22 is provided on the surface of the cover 2 which surface faces toward an affected area (lesion) 91 or is provided inside the cover 2. The cooling tank 22 is configured to contain a coolant (not illustrated) such as water or cold reserving material. The cooling tank 22 has the coolant inlet 23 through which the cooling tank 22 is in communication with the outside.

With the arrangement, even in a case where the light source unit 3 has generated heat during PDT (while the light source unit 3 is ON) and thereby the coolant in the cooling tank 22 has increased in temperature and the cooling effect has decreased, it is possible to feed a coolant through the coolant inlet 23 and possible to carry out replacement. With this, efficient heat exchange is achieved, making it possible to reduce the time required for cooling. This makes it possible to carry out PDT efficiently.

### Embodiment 7

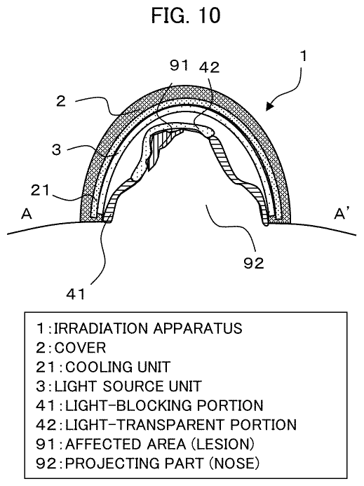

The following description will discuss Embodiment 7 of the present invention with reference to FIG. 10. For convenience of description, members having functions identical to those described in Embodiment 5 are assigned identical referential numerals, and their descriptions are omitted here. In Embodiment 7, differences from Embodiment 5 are discussed.

FIG. 10 is a cross-sectional view illustrating a configuration of an irradiation apparatus 1 in accordance with Embodiment 7. As illustrated in FIG. 10, the irradiation apparatus 1 includes a cover 2 and a light source unit 3, similarly to Embodiment 5. The irradiation apparatus 1 further includes a film that includes a light-blocking portion 41 and a light-transparent portion 42, instead of a sheet 4.

The light-blocking portion 41 is non-transparent to light emitted by the light source unit 3. The light-transparent portion 42 is transparent to the light emitted by the light source unit 3. The irradiation apparatus 1 may include the sheet 4 as necessary, similarly to Embodiment 5. The light-blocking portion 41 and the light-transparent portion 42 are arranged to cover an affected area (lesion) 91 and a projecting part (nose) 92. The light-transparent portion 42 is larger in size than the affected area (lesion) 91, is positionally in alignment with the affected area (lesion) 91, and covers the affected area (lesion) 91.

The light-transparent portion 42 is larger in size than the affected area (lesion) 91 for the following reason. When carrying out PDT, an agent containing δ-aminolevulinic acid is applied to the light-transparent portion 42. In so doing, the agent is applied so as to cover the affected area (lesion) with a margin of several millimeters, because there may be tissues with precancerous condition under the skin surrounding the apparent affected area (lesion). A photosensitizing substance is generated only in the area where the agent is applied, and lesion/abnormal tissues are destroyed by PDT.

On the other hand, since the photosensitizing substance is generated even in the surrounding skin where there are no tissues with precancerous condition under the skin (where there are no apparent tissues with precancerous condition), irradiation of this area with light of the PDT irradiation apparatus will cause pain or the like. That is, the agent needs to be applied only in the area of minimum necessary size.

Specifically, there can be an appropriate size for the area to be irradiated with the light of the PDT irradiation apparatus, in terms of achieving effective PDT.

The light-blocking portion 41 and the light-transparent portion 42 are prepared based on, for example, the results obtained by carrying out observation and measurement on-site. The light-blocking portion 41 and the light-transparent portion 42 may be prepared with a 3D printer with use of 3D data acquired using a 3D scanner.

### Embodiment 8

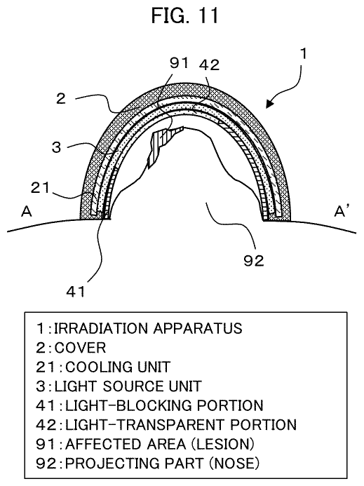

The following description will discuss Embodiment 8 of the present invention with reference to FIG. 11. For convenience of description, members having functions identical to those described in Embodiment 5 are assigned identical referential numerals, and their descriptions are omitted here. In Embodiment 8, differences from Embodiment 5 are discussed.

FIG. 11 is a cross-sectional view illustrating a configuration of an irradiation apparatus 1 in accordance with Embodiment 8. As illustrated in FIG. 11, the irradiation apparatus 1 includes a cover 2 and a light source unit 3, similarly to Embodiment 5. Embodiment 8 is different from Embodiment 7 in that a sheet 4 including a light-blocking portion 41 and a light-transparent portion 42 covers the light source unit 3, instead of the light-blocking portion 41 and the light-transparent portion 42 being put on an affected area (lesion) 91 and a projecting part (nose) 92. This embodiment is advantageous in that the similar effect to that achieved by Embodiment 7 can be achieved without having to touch the affected area (lesion) 91.

### Embodiment 9

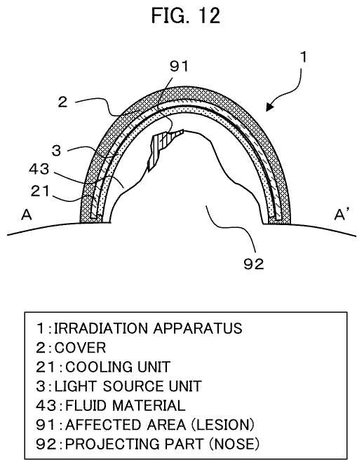

The following description will discuss Embodiment 8 of the present invention with reference to FIG. 12. For convenience of description, members having functions identical to those described in Embodiment 5 are assigned identical referential numerals, and their descriptions are omitted here. In Embodiment 9, differences from Embodiment 5 are discussed.

FIG. 12 is a cross-sectional view illustrating a configuration of an irradiation apparatus 1 in accordance with Embodiment 9. As illustrated in FIG. 12, the irradiation apparatus 1 includes a cover 2, a light source unit 3, a sheet 4, and a cooling unit 21, similarly to Embodiment 5. Embodiment 9 is different from Embodiment 5 in that the irradiation apparatus 1 further includes a fluid material 43 instead of the sheet 4.

The fluid material 43 fills the gap between (i) the light source unit 3 and (ii) an affected area (lesion) 91 and projecting part (nose) 92. In other words, the space enclosed by the light source unit 3 and the projecting part (nose) 92 is filled with the fluid material 43 so that the light source unit does not make contact with the affected area of the projecting part (nose) 92 or an area surrounding the affected area.

The fluid material 43 is preferably transparent to light emitted by the light source unit 3. The fluid material 43 is preferably composed of a material that is safe for a living body to touch. Examples of such a material include silicone resins for medical use, epoxy resins, styrene-based elastomer resins, and the like. Note, however, that the material is not limited to the resins listed above, provided that the material has similar characteristics to those of the resins listed above. The fluid material 43 makes it possible to reduce refraction when the light emitted by the light source unit 3 is released into the air. This makes it possible to more uniformly irradiate the affected area (lesion) 91 and the projecting part (nose) 92 with light of the irradiation apparatus 1.

The fluid material 43 may be formed by applying it onto the affected area (lesion) 91 and the projecting part (nose) 92 in advance and then attaching the irradiation apparatus 1. The irradiation apparatus 1 may include a means to form the fluid material 43, for example, a fluid material inlet and a fluid material outlet. In a case where the fluid material 43 in liquid state is not preferred, the fluid material 43 may be composed of a photo-curable resin, and may be cured with use of light emitted by the irradiation apparatus 1 or with use of some other light source.

### Embodiment 10

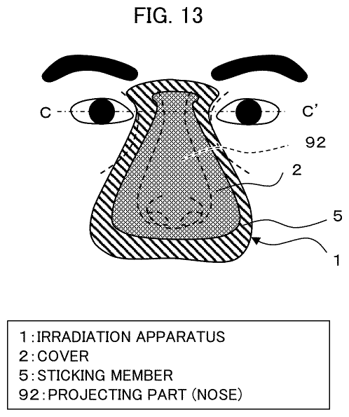

The following description will discuss Embodiment 10 of the present invention with reference to FIG. 13. For convenience of description, members having functions identical to those described with regard to Embodiment 1 are assigned identical referential numerals, and their descriptions are omitted here. In Embodiment 10, differences from Embodiment 1 are discussed.

FIG. 13 is a front view illustrating a configuration of an irradiation apparatus 1 in accordance with Embodiment 10. As illustrated in FIG. 13, the irradiation apparatus 1 includes a cover 2, a light source unit 3, and a sheet 4, similarly to Embodiment 1. The irradiation apparatus 1 further includes a sticking member 5. For convenience of illustration, the light source unit 3 and the sheet 4 are not illustrated. The following description will discuss each constituent of the irradiation apparatus 1 in more detail.

### Sheet 4

The sheet 4 is produced in the same manner as described in Embodiment 1. Unlike Embodiment 1, the sheet 4 is not necessarily in contact with a projecting part (nose) 92.

### Sticking Member 5

The sticking member 5 allows the irradiation apparatus to stick to the projecting part (nose) 92 or to an area surrounding the projecting part (nose) 92 (in the present specification, the projecting part (nose) 92 or its surrounding area may be referred to as “surrounding area”) through its stickiness. Note that the sticking member 5 can make contact also with an affected area (lesion) 91. The sticking member 5 is elastic at least in a part thereof which makes contact with the affected area (lesion) 91 or with the surrounding area.

The sticking member 5 is preferably composed of a material that is safe for a living body to touch. Examples of such a material include silicone resin for medical use, epoxy resin, styrene-based elastomer resin, and the like. Note, however, that the material is not limited to the resins listed above, provided that the material has similar characteristics to those of the resins listed above. In some cases, the sticking member 5 may have a multilayer structure whose surface only is composed of a material that is safe for a living body to touch.

The sticking member 5 is produced to conform to the size and shape of the projecting part (nose) 92. Specifically, the projecting part (nose) 92 can have various types corresponding to respective individuals or corresponding to groups in which individuals are categorized. The sticking member 5 is preferably non-transparent to light. Especially the nose is close to the eyes; therefore, care should be taken not to allow intense light emitted by the irradiation apparatus 1 to enter the eyes.

The sticking member 5 may be produced with use of a 3D printer or the like using 3D data acquired from the affected area (lesion) 91 and the projecting part (nose) 92 with a 3D scanner or the like. This makes it possible to produce a sticking member 5 most appropriate for the size of the projecting part (nose) 92, and possible to more appropriately prevent the intense light emitted by the irradiation apparatus 1 from entering the eyes.

### Embodiment 11

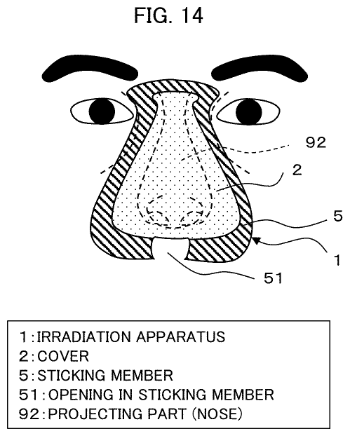

The following description will discuss Embodiment 11 of the present invention with reference to FIG. 14. For convenience of description, members having functions identical to those described in Embodiment 10 are assigned identical referential numerals, and their descriptions are omitted here. In Embodiment 11, differences from Embodiment 10 are discussed.

FIG. 14 is a front view illustrating a configuration of an irradiation apparatus 1 in accordance with Embodiment 11. As illustrated in FIG. 14, the irradiation apparatus 1 includes a cover 2, a light source unit 3, a sheet 4, and a sticking member 5, similarly to Embodiment 10. Embodiment 11 is different from Embodiment 10 in that the sticking member 5 has an opening 51 in a portion thereof. In other words, an area surrounding an affected area (lesion) 91, except for a portion thereof, is provided with the sticking member 5. For convenience of illustration, the light source unit 3 and the sheet 4 are not illustrated.

There may be a case in which, when the light source unit 3 is ON, the light source unit 3 generates heat and the heat is trapped in the space defined by the cover 2 and the sticking member 5. According to Embodiment 11, the sticking member 5 has the opening 51 in a portion thereof, which prevents the heat from being trapped. Furthermore, since the sticking member 5 sticks to the skin and is non-transparent to light, the light of the irradiation apparatus 1 cannot be seen by the irradiation target organism and medical staff in some cases. In such cases, whether PDT is carried out appropriately cannot be checked by the irradiation target organism and the medical staff. This is not preferred. Since there is the opening in the sticking member 5, the light of the irradiation apparatus 1 can be seen through the opening 51.

Note that the opening 51 in the sticking member 5 is preferably present at a position distant from the eyes of the irradiation target organism. This is because, if the opening 51 in the sticking member 5 is present near the eyes, intense light emitted by the irradiation apparatus 1 enters the eyes of the irradiation target organism. Therefore, the opening 51 in the sticking member 5 is preferably present on the opposite side of the sticking member 5 from where the sticking member 5 faces the eyes of the irradiation target organism. It is also preferable that a power supply line 13 (not illustrated) be connected to a power supply 12 through the opening 51 in the sticking member 5. There may be cases in which the light emitted by the light source unit 3 leaks out through the part through which the power supply line 13 is run outward; however, since the opening 51 in the sticking member 5 is present at a position distant from the eyes of the irradiation target organism, it is possible to prevent the light emitted by the light source unit 3 from entering the eyes of the irradiation target organism.

If the nose of the irradiation target organism is completely covered, breathing of the irradiation target organism will be greatly impaired. Therefore, by providing the opening 51 in the sticking member 5 near nostrils, it is possible to carry out PDT without impairing the breathing of the irradiation target organism.

The example shown in FIG. 14 can be expressed as follows: the surrounding area, except for a portion located away from the eyes of the irradiation target organism, is provided with the sticking member 5.

### Embodiment 12

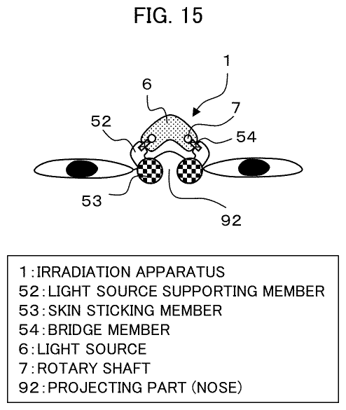

The following description will discuss Embodiment 12 of the present invention with reference to FIG. 15. For convenience of description, members having functions identical to those described in Embodiment 10 are assigned identical referential numerals, and their descriptions are omitted here. In this variation, differences from Embodiment 10 are discussed.

FIG. 15 is a cross-sectional view of a configuration of an irradiation apparatus 1 in accordance with Embodiment 12 in the vicinity of the eyes. Note that, in FIG. 15, a cover 2, a light source unit 3, and a sheet 4 are collectively illustrated as a light source 6. As illustrated in FIG. 15, the irradiation apparatus 1 includes the cover 2, the light source unit 3, the sheet 4, and a sticking member 5 (collectively referred to as the light source 6), similarly to Embodiment 10. The sticking member 5 is constituted by bridge members 54, light source supporting members 52, and skin sticking members 53. The sticking member 5 also includes rotary shafts 7. The following description will discuss each constituent of the irradiation apparatus 1 in more detail.

### Bridge Member 54

The bridge members 54 are connected to the rotary shafts 7 (each of which is provided in a part of the light source 6) and the light source supporting members 52. The bridge members 54 are preferably non-transparent to light, and can be made from, for example, a relatively rigid rubber.

### Light Source Supporting Member 52

The light source supporting members 52 are connected to the bridge members 54 and the skin sticking members 53. The light source supporting members 52 are preferably non-transparent to light, and can be made from, for example, a relatively rigid rubber.

### Skin Sticking Member 53

The skin sticking members 53 are each provided at one end of a corresponding light source supporting member 52, and stick to a projecting part (nose) 92. The skin sticking members 53 can be changed in shape as necessary. The skin sticking members 53 may have adhesiveness.

Embodiment 12 employs an arrangement in which (i) the bridge members 54, the light source supporting members 52, and the skin sticking members 53 make up the portion of the sticking member 5 which is closer to the eyes and (ii) the angle of each of the light source supporting members 52 relative to the light source 6 can be changed by rotation of the light source supporting member 52 about a corresponding rotary shaft 7. That is, the sticking member 5 includes, in each portion where the sticking member 5 connects to the cover 2, a rotary shaft 7 which has a range of motion that allows the relative angle between the cover and the projecting part (nose) 92 to change.

Each light source supporting member 52 is connected to a corresponding rotary shaft 7, and has a corresponding skin sticking member 53 at its opposite end from the rotary shaft 7. With this arrangement, the sticking member 5, which is constituted by the bridge members 54, the light source supporting members 52, and the skin sticking members 53, is non-transparent to light and is capable of preventing intense light emitted by the irradiation apparatus 1 from entering the eyes of an irradiation target organism and medical staff.

### Embodiment 13

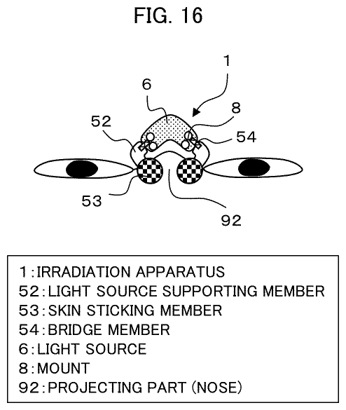

The following description will discuss Embodiment 13 of the present invention with reference to FIG. 16. For convenience of description, members having functions identical to those described in Embodiment 12 are assigned identical referential numerals, and their descriptions are omitted here. In Embodiment 13, differences from Embodiment 12 are discussed.

FIG. 16 is a cross-sectional view of a configuration of an irradiation apparatus 1 in accordance with Embodiment 13 in the vicinity of the eyes. Note that, in FIG. 16, a cover 2, a light source unit 3, and a sheet 4 are collectively illustrated as a light source 6, similarly to FIG. 15. As illustrated in FIG. 16, the irradiation apparatus 1 includes the cover 2, the light source unit 3 and the sheet 4 (collectively referred as the light source 6), and bridge members 54, light source supporting members 52, and skin sticking members 53 (collectively referred to as sticking member 5), similarly to Embodiment 12. The irradiation apparatus 1 further includes one or more mounts 8 instead of rotary shafts 7.

In this variation, the light source 6 includes the one or more mounts 8 for attachment of the sticking member 5. This makes it possible to replace the sticking member 5 with another sticking member 5. It is also possible to achieve the following: a plurality of sticking members 5 suitable for different shapes of projecting parts (noses) 92 in the vicinity of the eyes are prepared; one of the sticking members 5 suitable for the shape of the projecting part (nose) 92 of an irradiation target organism is attached on-site; and thereby intense light emitted by the irradiation apparatus 1 is more reliably prevented from entering the eyes of the irradiation target organism and medical staff.

### Embodiment 14

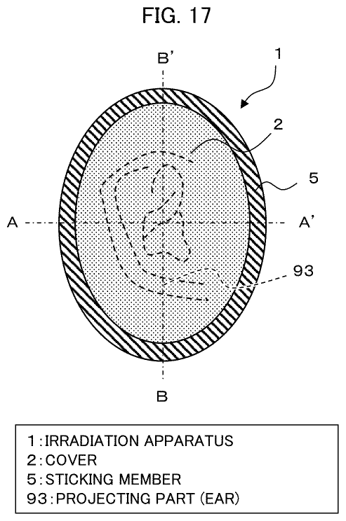

The following description will discuss Embodiment 14 of the present invention with reference to FIGS. 17 to 20. For convenience of description, members having functions identical to those described in Embodiment 10 are assigned identical referential numerals, and their descriptions are omitted here.

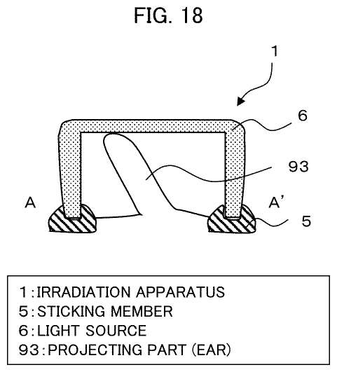

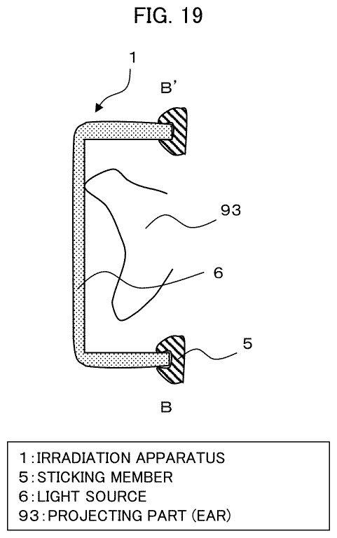

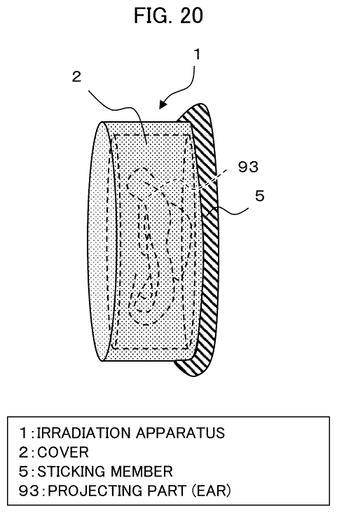

FIG. 17 is a top view illustrating a configuration of an irradiation apparatus 1 in accordance with Embodiment 14. FIG. 18 is a cross-sectional view illustrating the configuration of the irradiation apparatus 1 in accordance with Embodiment 14. FIG. 19 is a cross-sectional view illustrating the configuration of the irradiation apparatus 1 in accordance with Embodiment 14. FIG. 20 is a perspective view illustrating the configuration of the irradiation apparatus 1 in accordance with Embodiment 14.

FIG. 18 is a cross-sectional view of the irradiation apparatus 1 taken along line A-A′ in FIG. 17. Similarly, FIG. 19 is a cross-sectional view of the irradiation apparatus 1 taken along line B-B′ in FIG. 17. Note that, in FIGS. 18 and 19, a cover 2, a light source unit 3, and a sheet 4 are collectively illustrated as a light source 6.

The irradiation apparatus 1 in accordance with Embodiment 14 is suitable for, for example, a projecting part that has a plurality of faces, such as an ear having a front face and a back face.

As illustrated in FIGS. 17 to 20, the irradiation apparatus 1 includes the cover 2, the light source unit 3, and the sheet 4 (which are collectively illustrated as the light source 6), and a sticking member 5, similarly to Embodiment 7. The light source 6 is substantially in the shape of a top hat, i.e., a shape composed of (i) a substantially cylindrical shape which covers a projecting part (ear) 93 and (ii) a top panel which is in the shape of an oval and which is attached to the substantially cylindrical shape. The shape of the light source 6 is not limited to such a shape, and may be a polygon or the like.

As illustrated in FIG. 18, the projecting part (ear) 93 has three faces. The face facing left in FIG. 18, the face facing up in FIG. 18, and the face facing right in FIG. 18 can be irradiated with use of a single irradiation apparatus 1. In other words, the light source unit 3 is located such that the light source unit 3 is capable of irradiating the projecting part (ear) 93 with light from a plurality of directions.

### Embodiment 15

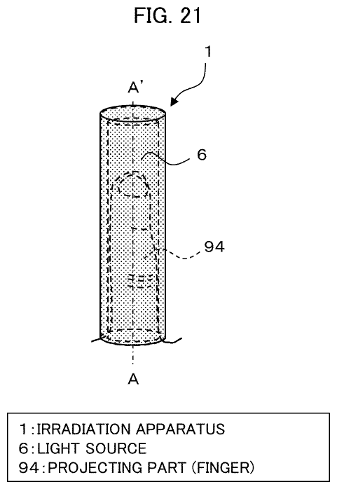

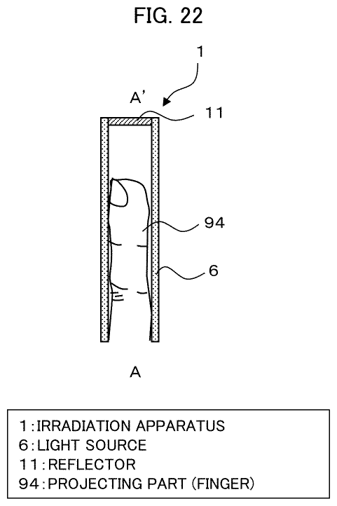

The following description will discuss Embodiment 15 of the present invention with reference to FIGS. 21 and 22. For convenience of description, members having functions identical to those described in Embodiment 10 are assigned identical referential numerals, and their descriptions are omitted here.

FIG. 21 is a perspective view illustrating a configuration of an irradiation apparatus 1 in accordance with Embodiment 15. FIG. 22 is a cross-sectional view illustrating the configuration of the irradiation apparatus 1 in accordance with Embodiment 15.

FIG. 22 is a cross-sectional view of the irradiation apparatus 1 taken along line A-A′ in FIG. 21. Note that, in FIGS. 21 and 22, a cover 2, a light source unit 3, and a sheet 4 are collectively illustrated as a light source 6. Also note that a sticking member 5 is not illustrated for convenience of illustration.

As illustrated in FIGS. 21 and 22, the irradiation apparatus 1 includes the cover 2, the light source unit 3, and the sheet 4 (which are collectively illustrated as the light source 6), and a sticking member 5, similarly to Embodiment 10. Embodiment 15 is different from Embodiment 10 in that the irradiation apparatus 1 further includes a reflector 11 which reflects light emitted by the light source unit 3 and which is provided so as to face a projecting part (finger) 94.

With regard to a finger, which is a kind of projecting part, an affected area (lesion) can develop at the tip of the finger. The projecting parts in the embodiments described so far are those having a certain size; however, the tip of a finger is only about 2 cm at the longest. It is difficult to place a light source unit 3 corresponding to such a small area.

In view of above, Embodiment 15 is arranged such that the reflector 11 such as silver is provided on a face which is substantially parallel to the tip of the finger and thereby the tip of the finger can be efficiently irradiated with light emitted by the light source unit 3. Note that, from that point of view, employing an LED mounted on the foregoing flexible substrate as the light source unit 3 is very useful, because the LED includes a reflector.

The cover 2 may be made up of a plurality of components which can be disassembled and assembled, not only in Embodiment 15 but also in the other embodiments. This can make it easy to attach the light source unit 3 to the cover 2.

The cover 2 may be configured to open with a part thereof connected to the light source unit 3.

### Embodiment 16

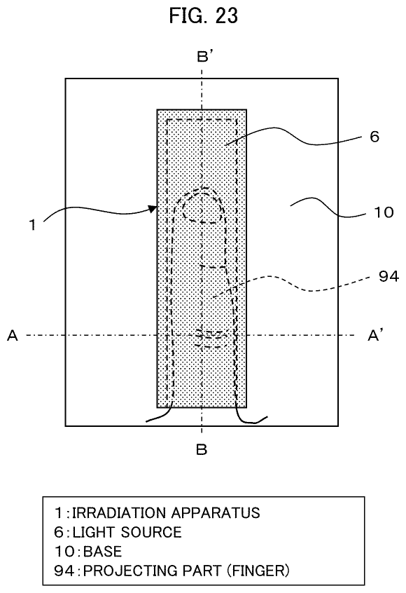

The following description will discuss Embodiment 16 of the present invention with reference to FIGS. 23 to 25. For convenience of description, members having functions identical to those described in Embodiment 15 are assigned identical referential numerals, and their descriptions are omitted here.

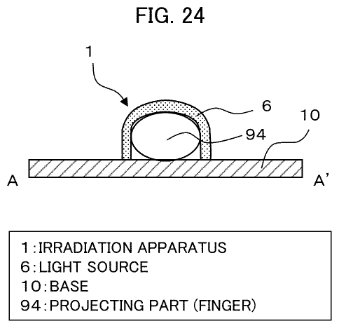

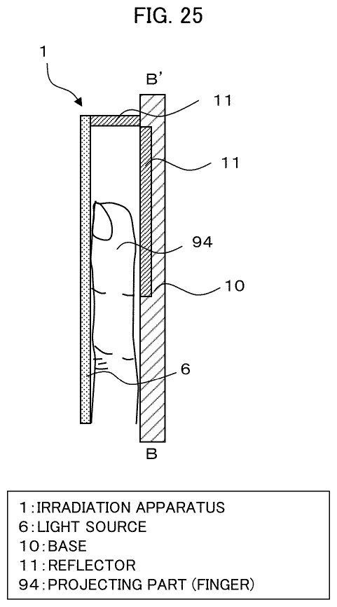

FIG. 23 is a top view illustrating a configuration of an irradiation apparatus 1 in accordance with Embodiment 16. FIG. 24 is a first cross-sectional view illustrating the configuration of the irradiation apparatus 1 in accordance with Embodiment 16. FIG. 25 is a second cross-sectional view illustrating the configuration of the irradiation apparatus 1 in accordance with Embodiment 16.

FIG. 24 is a cross-sectional view of the irradiation apparatus 1 taken along line A-A′ in FIG. 23. FIG. 25 is a cross-sectional view of the irradiation apparatus 1 taken along line B-B′ in FIG. 23. As illustrated in FIGS. 23 to 25, the irradiation apparatus 1 includes a cover 2, a light source unit 3, and a sheet 4 (which are collectively illustrated as light source 6), a sticking member 5, and a reflector 11, similarly to Embodiment 15. Embodiment 16 is different from Embodiment 15 in that the irradiation apparatus 1 further includes a base 10 for placement of a projecting part (finger) 94. The base 10 may have a recess corresponding to the shape of the projecting part (finger) 94.

Embodiment 16 makes it possible to effectively fix the projecting part (finger) 94. On the contrary, while Embodiment 16 is capable of irradiating, with light emitted by the irradiation apparatus 1, one side of the projecting part (finger) 94, it is incapable of irradiating, with the light, the opposite side of the projecting part (finger) 94.

As such, the irradiation apparatus 1 in accordance with Embodiment 16 further includes the base 10 that covers one side of the projecting part (finger) 94, and is arranged such that the cover 2 is located in a fixed position relative to the base 10 so as to cover another side(s) of the projecting part (finger) 94.

As illustrated in FIG. 25, the reflector 11 is provided not only on the face substantially parallel to the tip of the projecting part (finger) 94 but also on the base 10, for the following reason. The arrangement in FIG. 25 is for a person with a long projecting part (finger) 94; however, in a case where a person with a shorter projecting part (finger) 94 is to be irradiated with light emitted by the irradiation apparatus 1, the reflector 11 on the face substantially parallel to the tip of the projecting part (finger) 94 alone may be insufficient to irradiate the projecting part (finger) 94 with the light.

The cover 2 may be configured to swing open about a part thereof connected to the base 10.

### Embodiment 17

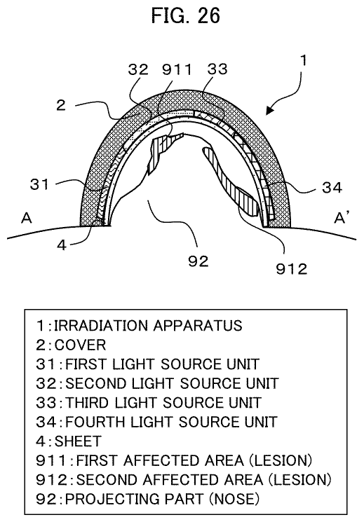

The following description will discuss Embodiment 17 of the present invention with reference to FIG. 26. For convenience of description, members having functions identical to those described in Embodiment 1 are assigned identical referential numerals, and their descriptions are omitted here.

FIG. 26 is a cross-sectional view illustrating a configuration of an irradiation apparatus 1 in accordance with Embodiment 17. The irradiation apparatus 1 in accordance with Embodiment 17 includes a cover 2, a light source unit 3, and a sheet 4, similarly to Embodiment 1. The light source unit 3 is constituted by a first light source unit 31, a second light source unit 32, a third light source unit 33, and a forth light source unit 34, which are controlled independently of one another.

As illustrated in FIG. 26, an affected area (lesion) 91 is made up of a first affected area (lesion) 911 and a second affected area (lesion) 912. The arrangement illustrated in FIG. 26 assumes a case in which a plurality of affected areas (lesions) 91 have developed on a projecting part (nose) 92.

The first light source unit 31, the second light source unit 32, the third light source unit 33, and the fourth light source unit 34, which are controlled independently of one another, can each be controlled in a manner corresponding to image data acquired by an image pickup device such as a CMOS imager installed in the irradiation apparatus 1.

Alternatively, the following arrangement may be employed: data or 3D scan data acquired in advance by checking an affected area is inputted; and thereby one or more necessary light source units 3 are selected automatically from a plurality of light source units 3.

Needless to say, the entirety of the projecting part (nose) 92 can be irradiated with light emitted by the irradiation apparatus 1. Alternatively, for example, (i) only the first affected area (lesion) 911, (ii) only the second affected area (lesion) 912, or (iii) both the first affected area (lesion) 911 and the second affected area (lesion) 912 can be irradiated with the light. This makes it possible to irradiate, with light, only the affected area at which irradiation is targeted and not to irradiate, with light, areas at which irradiation is not targeted, and also makes it possible to minimize power consumption.

### REFERENCE SIGNS LIST

1 irradiation apparatus

10 base

11 reflector

12 power supply

13 power supply line

2 cover

21 cooling unit

22 cooling tank

23 coolant inlet

3 light source unit

31 first light source unit

32 second light source unit

33 third light source unit

34 fourth light source unit

4 sheet

41 light-blocking portion

42 light-transparent portion

43 fluid material

5 sticking member

51 opening in sticking member

52 light source supporting member

53 skin sticking member

54 bridge member

6 light source

7 flexible LED

71 flexible substrate

72 front-side trace

73 reflector

74 light-emitting device (LED)

75 electrical conductor

76 sealant

77 connection hole

78 back-side trace

8 mount

91 affected area (lesion)

911 first affected area (lesion)

912 second affected area (lesion)

92 projecting part (nose)

93 projecting part (ear)

94 projecting part (finger)

1. An irradiation apparatus configured to irradiate a projecting part of a body of an irradiation target organism with light, the irradiation apparatus comprising:
a cover that corresponds in shape to the projecting part;
a light source unit that is superposed on the cover; and
a sheet that is provided so that the light source unit does not make contact with an affected area of the projecting part or an area surrounding the affected area and that is superposed on the cover with the light source unit interposed between the sheet and the cover,
the sheet and/or the cover being configured to make contact with the area surrounding the affected area.

2. The irradiation apparatus as set forth in claim 1, wherein the light source unit includes:
an insulating flexible substrate;
a trace provided on the insulating flexible substrate; and
a light-emitting device mounted on the trace.

3. The irradiation apparatus as set forth in claim 1, wherein the sheet partially covers the cover so that the cover does not make contact with the projecting part.

4. The irradiation apparatus as set forth in claim 1, wherein the sheet covers both faces of the cover.

5. The irradiation apparatus as set forth in claim 1, further comprising a cooling unit that is configured to cool the light source unit and that is superposed on at least one face of the cover

6. The irradiation apparatus as set forth in claim 5, wherein the cooling unit is in contact with at least one face of the light source unit and is configured to exchange heat with the light source unit.

7. The irradiation apparatus as set forth in claim 5, wherein the cooling unit is flexible and at least one face of the cooling unit has adhesiveness.

8. The irradiation apparatus as set forth in claim 5, wherein the cooling unit has a coolant inlet which allows a coolant to be fed externally.

9. The irradiation apparatus as set forth in claim 1, wherein the sheet includes:
a light-transparent portion that is transparent to light emitted by the light source unit; and
a light-blocking portion that is non-transparent to the light emitted by the light source unit.

10. An irradiation apparatus configured to irradiate a projecting part of a body of an irradiation target organism with light, the irradiation apparatus comprising:
a cover that corresponds in shape to the projecting part; and
a light source unit that is superposed on the cover, wherein
a space enclosed by the light source unit and the projecting part is filled with a fluid material so that the light source unit does not make contact with an affected area of the projecting part or an area surrounding the affected area, and
the cover is configured to make contact with the area surrounding the affected area.

11. An irradiation apparatus configured to irradiate a projecting part of a body of an irradiation target organism with light, the irradiation apparatus comprising:
a cover that corresponds in shape to the projecting part;
a light source unit that is superposed on the cover;
a sheet that is provided so that the light source unit does not make contact with an affected area of the projecting part or an area surrounding the affected area and that is superposed on the cover with the light source unit interposed between the sheet and the cover; and
a sticking member that is configured to allow the irradiation apparatus to stick to the area surrounding the affected area,
the sticking member being elastic at least in a part thereof which makes contact with the area surrounding the affected area.

12. The irradiation apparatus as set forth in claim 11, wherein the sticking member is configured such that the area surrounding the affected area, except for a portion thereof, is provided with the sticking member.

13. The irradiation apparatus as set forth in claim 12, wherein the sticking member is configured such that the area surrounding the affected area, except for a portion located away from eyes of the irradiation target organism, is provided with the sticking member.

14. The irradiation apparatus as set forth in claim 11, wherein the sticking member includes, in a portion where the sticking member connects to the cover, a rotary shaft which has a range of motion that allows a relative angle between the cover and the projecting part to change.

15. The irradiation apparatus as set forth in claim 11, wherein the cover includes one or more mounts for attachment of the sticking member.

16. The irradiation apparatus as set forth in claim 1, wherein the light source unit is located such that the light source unit is capable of irradiating the projecting part with light from a plurality of directions.

17. The irradiation apparatus as set forth in claim 1, further comprising a reflector which is configured to reflect light emitted by the light source unit and which is provided so as to face the projecting part.

18. The irradiation apparatus as set forth in claim 1, further comprising a base that covers one side of the projecting part,
the cover being located in a fixed position relative to the base so as to cover another side of the projecting part.

19. The irradiation apparatus as set forth in claim 1, wherein the light source unit is composed of a plurality of independent light source units.

20. A method of irradiation, comprising using an irradiation apparatus which is configured to irradiate a projecting part of a body of an irradiation target organism with light, the method comprising the steps of:
putting an agent-containing film over an affected area of the projecting part and an area surrounding the affected area; and
carrying out irradiation with light,
the irradiation apparatus including
a cover that corresponds in shape to the projecting part and
a light source unit that is superposed on the cover, the agent-containing film including
a light-transparent portion that is configured to cover the affected area, that is larger in size than the affected area, and that is transparent to light emitted by the light source unit, and
a light-blocking portion that is non-transparent to the light emitted by the light source unit.
